(19) United States
(12) Patent Application Publication
(10) Pub. No.: US 2017/0086907 A1
(43) Pub. Date: Mar. 30, 2017

(54) RADIOFREQUENCY BALLOON CATHETER SYSTEM

(71) Applicant: Japan Electel Inc., Kanagawa (JP)

(72) Inventor: Shutaro SATAKE, Kanagawa (JP)

(73) Assignee: Japan Electel Inc., Kanagawa (JP)

(21) Appl. No.: 15/276,261

(22) Filed: Sep. 26, 2016

(30) Foreign Application Priority Data
Sep. 28, 2015 (JP) 2015-190313

(51) Int. Cl. A61B 18/12; A61B 18/14

(52) U.S. Cl. CPC A61B 18/12; A61B 18/1206; A61B 18/1492; A61B 2018/0022

(57) ABSTRACT

A radiofrequency balloon catheter system to heat and dilate a stenosis site while protecting an intima. The system includes an elastic balloon with its anterior neck covering an inner tube, and the anterior neck has a distal portion fixed to the inner tube and a proximal portion contacted by the inner tube to thereby define a check valve. When an in-balloon solution is suctioned to turn the inside pressure of the balloon to negative, the check valve is closed to deflate the balloon. When the in-balloon solution is injected into the balloon to turn the inside pressure of the balloon to positive, the balloon is inflated and the check valve is opened, thus discharging the solution to the outside via small holes bored through a specific portion of the inner tube that serves as a valve seat.

### CROSS-REFERENCE TO RELATED APPLICATIONS

This application claims priority to Japanese Patent Application No. 2015-190313, filed Sep. 28, 2015, the entire contents of which are incorporated herein by reference.

### TECHNICAL FIELD

The present invention relates to a radiofrequency balloon catheter system for thermally dilating a stenosis by inserting a deflated balloon into the stenosis within a hollow organ, and irradiating the stenosis with a radiofrequency electric field power via an internal electrode while applying a pressure to the balloon, with an intima being protected by perfusing the inside of the balloon with a coolant.

### BACKGROUND ART

Many of stenoses, such as coronary artery stenosis that cause angina or myocardial infarction are known to be due to arteriosclerotic lesions in a vascular membrane, and hence such stenoses are improved if they are dilated while applying a heat thereto using a radiofrequency hot balloon catheter. One example of ablation systems using such radiofrequency hot balloon catheter is disclosed in e.g., Japanese Unexamined Patent Application Publication No. 2002-126096.

According to conventional radiofrequency hot balloon catheters, the balloon is deflated and inserted into a vascular stenosis site, then the balloon is pressurized and inflated to dilate the vascular stenosis, while heating the site by applying thereto a radiofrequency energy from an electrode inside the balloon to fuse collagen tissues and atheroma, etc. therein. Whilst such method as to dilate a vessel at a relatively low pressure while heating the vessel to soften and fuse a lesion therein has an advantage that the method does not cause vascular dissociation or recoil, and hence it is free from a risk of developing acute obstruction. The method, however, has a problem that restenosis may occur due to intimal proliferation caused by intimal ablation.

In order to prevent damages to an intima of a blood vessel, there have been developed balloon cooling methods using perfusion inside a balloon. Such methods include a method of perfusing a balloon interior through an outer tube and an inner tube of a catheter shaft, as disclosed in U.S. Pat. No. 6,952,615, and a method of performing perfusion between the inside and the outside of a balloon through pores in a balloon film, as disclosed in U.S. Pat. No. 6,491,710, both of which were invented by the inventor of the present invention. Other balloon fluid discharge mechanisms developed by other inventors are disclosed in, e.g., Japanese Unexamined Patent Application Publication Nos. 2011-526820 and 2000-508197.

Among the systems to prevent damages to an intima of a blood vessel through an in-balloon perfusion system during thermal angioplasty using a radiofrequency balloon catheter, the system of U.S. Pat. No. 6,952,615 perfusing the inside of a balloon through the outer and inner tubes of a catheter shaft, exhibits an insufficient balloon cooling capacity due to a comparatively small amount of a perfusing solution resulting from a narrow shaft lumen for such a thin catheter as is used for coronary artery, etc.

According to the perfusion system of U.S. Pat. No. 6,491,710 that discharges an in-balloon solution to the exterior through pores in a balloon film, passages between the balloon and the exterior are always open, thus making it impossible to adjust its perfusion rate, and hence even if the solution is strongly suctioned from the inside of the balloon, the balloon does not fully deflate, making it difficult to insert the balloon catheter into a vascular stenosis site.

The aforesaid Japanese Unexamined Patent Application Publication Nos. 2011-526820 and 2000-508197 also disclose an in-balloon perfusion mechanism, in which a solution discharged from the inside of the balloon passes through a gap between a balloon neck and an inner tube thereof, and yet a volume of discharge depends on a pressure inside the balloon and is not capable of being independently fine-adjusted, while the distal end of the balloon neck is not fixed to the inner tube, resulting in a stepped portion being formed therebetween, posing an obstacle to the passage through a severe stenosis portion of a blood vessel, and if the catheter is forced therethrough, there may be caused a deformation of the catheter tip. It is to be noted herein that neither of these related arts includes a radiofrequency heating function added thereto.

### SUMMARY OF THE INVENTION

In view of the problems described above, the balloon catheter system of the present invention employs a perfusion system with an enhanced balloon cooling capacity that discharges a solution within a balloon to the outside thereof, in which a distal end of the balloon neck is fixed to the distal end of the inner tube, and small holes are bored in the front part of the inner tube. Then, a perfusate is allowed to pass through a gap between an anterior neck of the balloon and the front part of the inner tube, and then discharged from the distal end of the inner tube of the catheter through small holes bored in the front part of the inner tube, or otherwise, it is allowed to pass through a gap between the small holes provided in the front part of the inner tube and a guide wire coated with a resilient material, and then discharged from the distal end of the inner tube. According to the balloon catheter system of the present invention, it is designed such that volume of discharge is adjusted by moving in and out the guide wire loaded into the inner tube.

Accordingly, it is an object of the present invention to provide a radiofrequency balloon catheter system such that without the need to change a conventional profile of balloon, the balloon is deflated by suctioning the balloon with the distal end of the anterior neck of the balloon being fixed to the distal end of the inner tube of the catheter, thus enabling the balloon catheter to be easily passed through a severe stenosis site of a blood vessel to thereby heat and dilate the stenosis site at a moderate pressure, while protecting an intima by fine-adjusting a perfusion rate of a coolant within the balloon.

### MEANS FOR SOLVING THE PROBLEMS

According to the radiofrequency balloon catheter system of the present invention, the anterior neck of the balloon has a distal portion fixed to the inner tube and a proximal portion contacted by the inner tube to thereby define a check valve; and small holes are bored through a part of said inner tube that serves as a valve seat for said check valve.

Accordingly, when a solution is injected into the balloon, the pressure within the balloon turns to positive to thereby inflate the balloon, thereby opening the valve defined by the proximal portion of the anterior neck of the balloon and the inner tube, followed by discharge of the solution to the outside through the small holes bored in the inner tube to thereby cool the balloon interior.

When the solution is suctioned from the balloon to turn the pressure within the balloon to negative, then the valve is closed and then the balloon is deflated. At this time, since the proximal portion of the anterior neck of the balloon is fixed to the inner tube, insertion into the stenosis site becomes even easier, and the perfusate rate becomes adjustable through in-and-out operation of the guide wire loaded into the inner tube, thus providing a solution to the above-described problems.

Since the present balloon cooling system utilizes conventional balloon catheter members, the system can be also applied to small diameter catheters such as those for coronary angioplasty without changing the balloon profile.

According to a first aspect of the present invention, there is provided a radiofrequency balloon catheter system including:

a catheter shaft comprising an inner tube and an outer tube;

a resilient balloon that is inflatable and deflatable and provided between distal ends of the inner tube and the outer tube, said balloon including an anterior neck covering said inner tube, said anterior neck having a distal portion fixed to said inner tube and a proximal portion contacted by said inner tube to thereby define a check valve;

one or more transmural small holes bored through a part of said inner tube that serves as a valve seat for said check valve;

an electrode for delivery of radiofrequency current provided within the balloon;

a radiofrequency generator connected to the electrode for delivery of radiofrequency current via a connecting wire within said catheter shaft;

a solution transport path defined by the outer tube and the inner tube, said solution transport path being in communication with an inside of the balloon, and connected to a liquid feed pump for feeding a coolant; and

a guide wire insertable into said inner tube, as illustrated in FIGS. 1 to 4.

According to a second aspect of the present invention, there is provided a radiofrequency balloon catheter system including:

a catheter shaft comprising an inner tube and an outer tube;

a resilient balloon that is inflatable and deflatable and provided between distal ends of the inner tube and the outer tube, said balloon including an anterior neck fixed to said inner tube;

one or more small holes bored through a distant portion of said inner tube within said balloon;

a guide wire provided within said inner tube, said guide wire having a surface coated with a resilient material;

a check valve defined by said small holes and said guide wire being contacted by each other;

an electrode for delivery of radiofrequency current provided within the balloon;

a radiofrequency generator connected to the electrode for delivery of radiofrequency current via a connecting wire within said catheter shaft; and

a solution transport path defined by the outer tube and the inner tube, said solution transport path being in communication with an inside of the balloon, and connected to a liquid feed pump for feeding a coolant, as illustrated in FIGS. 5A to 5C.

According to the radiofrequency balloon catheter system of any of the foregoing aspects, the number of said one or more transmural small holes is preferably 1 to 10.

According to the radiofrequency balloon catheter system of any of the foregoing aspects, said guide wire preferably has such a tapered distal end that conforms to a lumen of said inner tube.

According to the radiofrequency balloon catheter system of any of the foregoing aspects, a temperature sensor for measurement of a perfusate temperature is preferably attached to the distal end of said inner tube, as illustrated in FIG. 6.

According to the radiofrequency balloon catheter system of any of the foregoing aspects, it is preferable that a temperature sensor and a pressure sensor are installed within said balloon and are respectively connected to a temperature measurement device and a pressure measurement device via a connecting wire, as illustrated in FIG. 6.

According to the radiofrequency balloon catheter system of any of the foregoing aspects, it is preferable that an electrode is installed in front and back of said balloon on said catheter shaft, and the electrode is connected to an impedance measurement device via a connecting wire, as illustrated in FIG. 6.

According to the radiofrequency balloon catheter system of any of the foregoing aspects, said balloon is made up of a film that is preferably either a conductive film or a porous film,

Referring to FIG. 1A showing a schematic diagram of the present invention in accordance with the first aspect of the invention, when a coolant is injected into the inside of the balloon via the catheter shaft, the balloon is inflated so that the check valve defined by the proximal portion of the anterior neck and the inner tube is opened, thereby allowing the coolant to be discharged from the distal end of the inner tube to the outside through the small holes bored through the inner tube. At this time, if the guide wire is inserted deep enough up to the distal end of the inner tube, then, a route of discharge is subjected to an increased resistance, resulting in a decreased discharge rate of the coolant, as shown in FIG. 1B. When the solution within the balloon is suctioned, the balloon is then deflated so that the check valve defined by the proximal portion of the anterior neck and the inner tube is closed, thereby allowing the flow of the coolant to be interrupted, thus turning the pressure inside the balloon to negative, as shown in FIG. 1C. When the solution within the balloon is further suctioned, the balloon is deflated small enough to be easily inserted into the stenosis site, as shown in FIG. 2.

Upon delivery of radiofrequency current, a radiofrequency electric field is radiated uniformly from the electrode for delivery of radiofrequency current, thereby allowing the balloon to dilate the stenosis while heating the same, and if a coolant is injected into the balloon simultaneously therewith, the check valve is opened, allowing the coolant to be discharged from the lumen of the inner tube to the outside via the small holes of the inner tube serving as a valve seat, thus cooling the balloon, as illustrated in FIGS. 3 and 4. At this time, the discharge rate of the coolant from the distal end of the catheter is adjustable through the manipulation of the guide wire. If the guide wire is inserted up to the distal end of the inner tube, the route of discharge is subjected to an increased resistance, resulting in a decreased discharge rate of the coolant, while if the guide wire is pulled backwardly of the distal end of the inner tube, the route of discharge is subjected to a decreased resistance, resulting in an increased discharge rate of the coolant.

According to the first aspect of the invention, there can be provided a radiofrequency balloon catheter system enabling a balloon catheter thereof to easily pass through a stenosis and dilate the stenosis while heating the same, with an intima being protected by a cooling effect achieved by an appropriate perfusion inside the balloon.

The system according to the second aspect of the invention is such that a discharge route for the coolant is ensured by boring the small holes through the inner tube within the balloon so that the inner tube may serve as a valve seat, while the guide wire having such a resiliency that dilates and contracts in response to a pressure is allowed to serve as a valving element, whereby the check valve is closed to deflate the balloon when the inside of the balloon is under negative pressure, while the check valve is opened to discharge the coolant when the inside of the balloon is under positive pressure, as illustrated in FIGS. 5A to 5C. Like in the first aspect of the invention, there can be provided a radiofrequency balloon catheter system enabling a stenosis to be dilated while heating the same, with an intima being protected through the perfusion inside the balloon while delivering a radiofrequency current thereto.

According to one of the preferred embodiments of the foregoing aspects of the present invention, the amount of a coolant to be perfused within the balloon is capable of being fine-adjusted, by increasing the number of the small holes bored through the inner tube that serve as a valve seat.

According to another preferred embodiment thereof, the guide wire, having a role to adjust the discharge rate of a coolant by closing the lumen of the inner tube, has such a tapered distal end that conforms to the lumen of said inner tube, thereby enhancing the function thereof.

According to a further preferred embodiment thereof, a temperature sensor for measurement of a perfusate temperature is attached to the distal end of the inner tube, thus making it possible to measure a temperature of a perfusate discharged from the catheter. If the temperature is kept at 45 degrees C. or below, it is possible to reduce peripheral vascular disorder to minimum, while if the temperature is kept at more than 45 degrees C., it is possible to perform hyperthermic treatment to a peripheral perfusion area.

According to a still further preferred embodiment thereof, a temperature sensor and a pressure sensor are installed within the balloon, making it possible to monitor a balloon temperature and a pressing force of the balloon against tissues, thereby enabling one to make sure that ablation of a target tissue has been successfully done.

According to yet another preferred embodiment thereof, an electrode is installed in front and back of the balloon, making it possible to monitor an impedance around the balloon, thereby enabling one to follow up the extent of ablation of a target tissue.

According to a further preferred embodiment thereof, the electrical conductivity of the balloon film is enhanced, thus facilitating the emission of a radiofrequency field to surrounding tissues.

### BRIEF DESCRIPTION OF THE DRAWING

FIG. 1A is an explanatory drawing illustrating a main framework of a radiofrequency balloon catheter system of the present invention, in which a balloon is additionally provided, at its distal end, with a check valve structure for an in-balloon perfusion system defined by a balloon anterior neck and an inner tube with small holes bored therethrough, such that the balloon is inflated if an in-balloon solution is injected thereinto, and the in-balloon solution is discharged from the inside of the balloon to the outside thereof through a space between the balloon anterior neck and the inner tube, and small holes of the inner tube.

FIG. 1B is an explanatory drawing illustrating a mechanism for adjusting a discharge rate of an in-balloon solution through a manipulation of a guide wire according to a radiofrequency balloon catheter system of the present invention.

FIG. 1C is an explanatory drawing illustrating a mechanism for deflating a balloon due to a closure of a valve defined by the balloon anterior neck and the inner tube when suctioning an in-balloon solution according to a radiofrequency balloon catheter system of the present invention.

FIG. 2 is an explanatory drawing illustrating a balloon catheter being inserted into a stenosis using a guide wire after the balloon is deflated by strongly suctioning the inside of the balloon.

FIG. 3 is an explanatory drawing illustrating the stenosis being heated by irradiation of a radiofrequency field while allowing the inside of the balloon to be perfused with a coolant, after delivery of a radiofrequency current is started with the balloon being inflated by injecting the coolant thereinto.

FIG. 4 is an explanatory drawing illustrating the stenosis being fully dilated by further increasing an in-balloon pressure by raising an injection speed of the coolant.

FIG. 5A is an explanatory drawing illustrating a main framework where the inner tube within the balloon has a nozzle at its distal end such that a valve is formed by the contact between the inner tube and a resilient portion of the guide wire such that the balloon is inflated if an in-balloon solution is injected thereinto with the distal end of the guide wire being located in a posterior position to thereby discharge the solution to the outside through the small holes of the inner tube.

FIG. 5B is an explanatory drawing illustrating such main framework in which with the distal end of the guide wire being located in an anterior position, the resilient portion of the guide wire is contracted when the inside of the balloon is under positive pressure to thereby discharge the solution through the space between the guide wire and the inner tube.

FIG. 5C is an explanatory drawing illustrating such main framework in which with the distal end of the guide wire being located in an anterior position, the resilient portion of the guide wire is expanded when the inside of the balloon is under negative pressure to thereby allow the guide wire to close the nozzle of the inner tube so that the balloon is deflated.

FIG. 6 is an explanatory drawing illustrating another framework where a temperature sensor and a pressure sensor are installed at distal portions of the inner tube, thus enabling the measurement of a temperature of the in-balloon solution and a pressure inside the balloon, while an electrode is attached to the vicinity of the distal end of the inner tube and the outer tube, thus enabling the measurement of an impedance across the front and rear of the balloon.

### MODE FOR CARRYING OUT THE INVENTION

As follows is a detailed description of embodiments of a radiofrequency balloon catheter system proposed by the present invention with reference to the appended drawings.

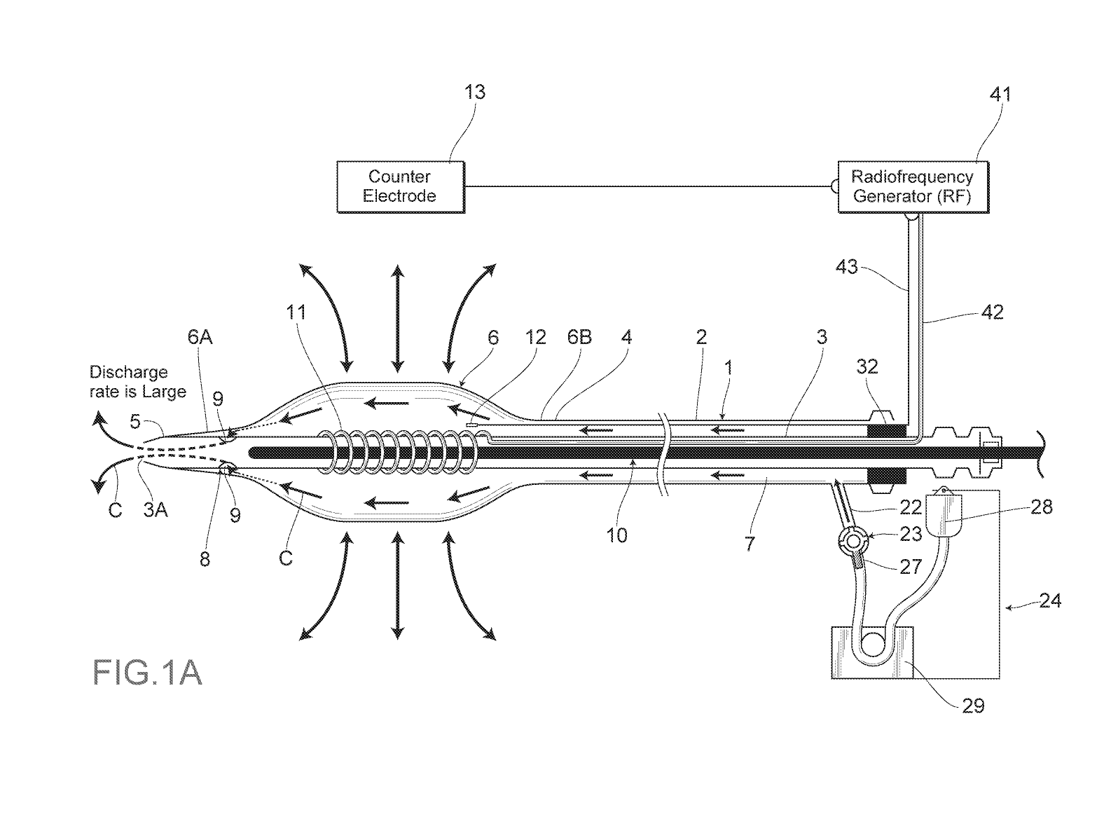

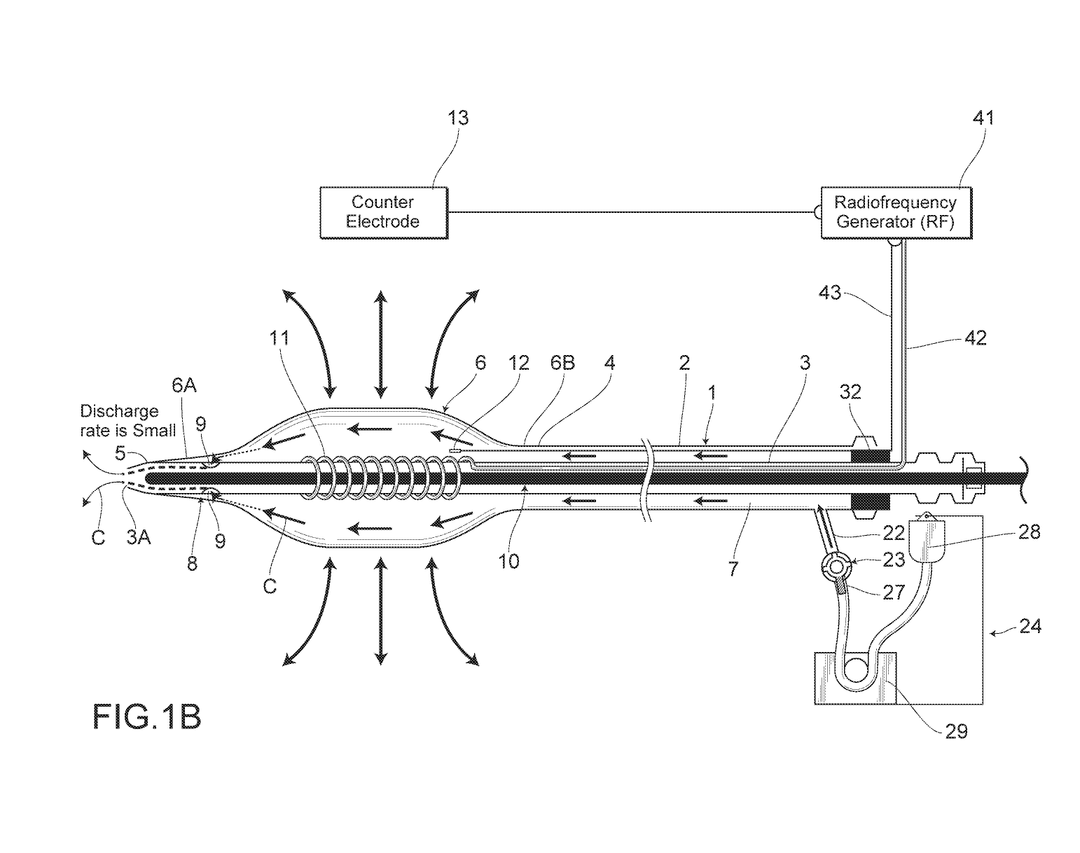

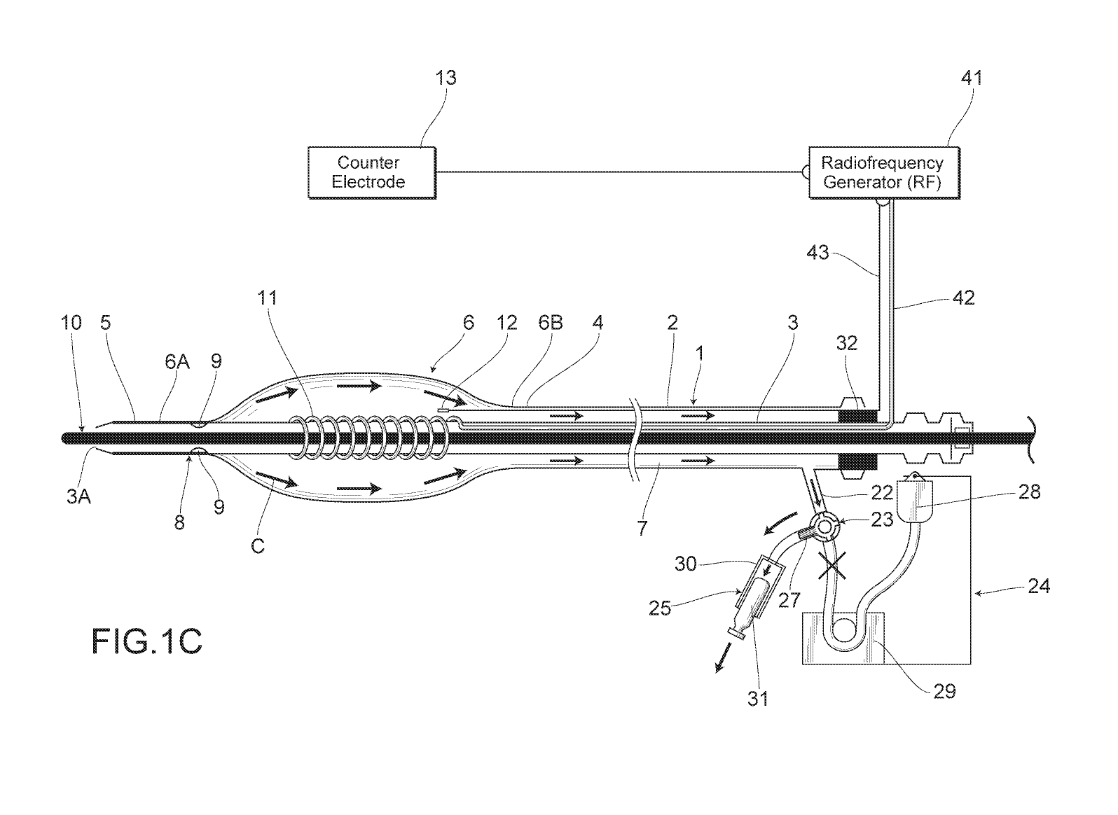

FIGS. 1A to 1C illustrate a major part structure of the radiofrequency balloon catheter system according to an embodiment of the present invention. In the drawings, numerical symbol 1 denotes a cylindrical catheter shaft that is rich in elasticity and insertable into a luminal organ. The catheter shaft 1 includes an outer tube 2 and an inner tube 3 which are hollow. A deflatable and inflatable balloon 6 is provided between a distal end 4 of the outer tube 2 and a vicinity of a distal end 5 of the inner tube 3. The balloon 6 is made of a thin membrane, which is formed of a heat-resistant resin such as polyurethane, PET (polyethylene terephthalate) or the like. The balloon 6 has an appropriate elasticity, and contains necks 6A and 6B respectively arranged in the anterior and posterior portions of the balloon 6. The necks 6A and 6B are comparatively long and have a thickness thinner than any other portions of the balloon. The balloon 6 is allowed to inflate in the shape of a rotating body, e.g., substantially spherical shape, by filling a solution as a coolant C (normally, a cooled mixture of a contrast agent and one of physiological saline and dextrose in water) in the balloon 6.

Between the outer tube 2 and the inner tube 3 is defined a solution transport path 7 in communication with the inside of the balloon 6. The anterior neck 6A of the balloon 6 has a distal portion provided as a distal end section thereof fixed to the inner tube 3, while a proximal portion provided as a proximal end section of the anterior neck 6A is not fixed to the inner tube 3 but is contacted by an outer surface of the inner tube 3 to thereby define a check valve 8. In the distal side portion of the inner tube 3 serving as a valve seat for the check valve 8, small holes 9 for discharging the fluid therefrom are bored through a sidewall of the inner tube 3. As illustrated in FIG. 1A, when the balloon 6 is subjected to a positive pressure, the anterior neck 6A movers away from the inner tube 3 to thereby open the small holes 9, thus forming a solution discharge route for allowing the coolant C to be discharged from the inside of the balloon 6 to the outside thereof. In contrast to that, as illustrated in FIG. 1C, when the balloon 6 is subjected to a negative pressure, the anterior neck 6A is deformed and comes in contact with the inner tube 3 to thereby close the small holes 9. In this way, the anterior neck 6A serves as a valving element of the check valve 8 for unidirectionally blocking the flow of the coolant C. Also, the inner tube 3 serves as a valve seat of the check valve 8. Meanwhile, the posterior neck 6B of the balloon 6 is fixed to or continuously provided on the distal end portion 4 of the outer tube 2. Numeral 10 denotes a guide wire for guiding the balloon 6 to a target site. The guide wire 10 is provided within the inner tube 3 in a manner extending therethrough.

At a tip of the distal end portion of the inner tube 3 is provided a discharge hole 3A for discharging the coolant C that has reached the inner route of the inner tube 3 through the small holes 9, to the outside of the inner tube 3. The proximal end portion of the inner tube 3, in contrast, is sealed to prevent leakage of the coolant C in a basal end side of the catheter shaft 1. The discharge hole 3A provided as a distal end aperture of the inner tube 3 is shaped such that the guide wire 10 is allowed to be inserted therethrough.

Inside the balloon 6 are arranged an electrode 11 for delivery of radiofrequency current and a temperature sensor 12. The electrode 11 for delivery of radiofrequency current is arranged, as an electrode for radiating a radiofrequency electric field, in such a coiled fashion that it is wound around the inner tube 3. Further, the electrode 11 for delivery of radiofrequency current has a monopolar structure, and is able to deliver a radiofrequency current between itself and a counter electrode 13 provided outside the catheter shaft 1. When a current is applied thereto, then, there will be radiated an electric field from the electrode 11 for delivery of radiofrequency current to the surroundings thereof.

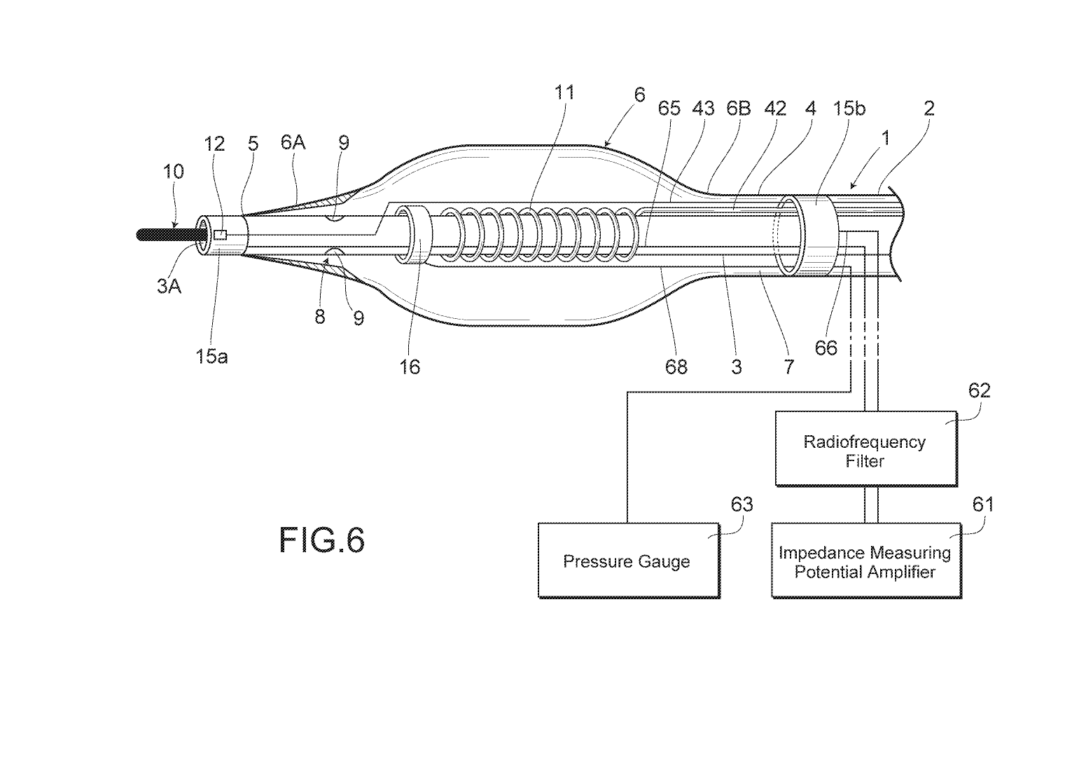

A temperature sensor 12, serving as a temperature detection unit, is provided on the proximal end side of the inner tube 3 inside the balloon 6, and arranged adjacent to the electrode 11 for delivery of radiofrequency current to detect the temperature thereof. Further, as illustrated in FIG. 6, there can be fixed not only the temperature sensor 12 but also electrodes 15a, 15b that are respectively provided on the anterior and posterior portions of the balloon 6 in order to measure the impedance therebetween. Further, in proximity to a front surface of the membrane inside the balloon 6, there may be provided a high directional pressure sensor 16 coaxially with the catheter shaft 1 with an input surface thereof facing forward in a longitudinal direction of the shaft 1.

Outside the catheter shaft 1, a communication tube 22 is connected to a basal portion of the solution transport path 7 in a communicative manner. One port of a three-way cock 23 is coupled to the basal portion of this communication tube 22, and the remaining two ports of the three-way cock 23 are respectively coupled to a liquid transfusing unit 24 for inflating the balloon 6 and a syringe 25 for deflating the balloon 6. The three-way cock 23 has an operation piece 27 capable of being pivotally operated by the fingers such that one of the liquid transfusing unit 24 and the syringe 25 may come into a fluid communication with the communication tube 22, or eventually with the solution transport path 7 by the operation of the operation piece 27.

The liquid transfusing unit 24 is made up of: an infusion bottle 28 for reserving the coolant C; and a liquid transfusing pump 29 in communication with the infusion bottle 28. When the liquid transfusing pump 29 is activated with the liquid transfusing unit 24 and the communication tube 22 communicated with each other through the three-way cock 23, the coolant C, having reached there from the infusion bottle 28, is pumped out into the solution transport path 7 through the liquid transfusing pump 29, thereby turning the pressure at the inside of the balloon 6 to positive. A syringe 25, serving as a liquid recovering unit, includes a cylindrical body 30 connected to the three-way cock 23 and a movable plunger 31 provided within the cylindrical body 30. If the plunger 31 is pulled back with the syringe 25 being communicated with the communication tube 22 through the three-way cock 23, the solution is recovered from the inside of the balloon 6 via the solution transport path 7 into the inside of the cylindrical body 30, thereby turning the pressure inside the balloon to negative. Between the outer tube 2 and the inner tube 3 is arranged a plug 32 for closing an aperture provided at the basal end side of the catheter 1 in order to prevent the occurrence of leakage of the coolant C therefrom while the coolant C is flowing.

Further, a radiofrequency generator 41 is provided outside of the catheter shaft 1. Within the balloon 6 are arranged the electrode 11 for delivery of radiofrequency current and the temperature sensor 12, which are electrically connected to the radiofrequency generator 41 respectively through the electric wires 42, 43 placed inside the catheter shaft 1. The radiofrequency generator 41 supplies a radiofrequency energy, to be delivered as an electric power, to between the electrode 11 for delivery of radiofrequency current and the counter electrode 13 through the electric wire 42, and heats the whole of the balloon 6 filled with the solution. The radiofrequency generator 41 is provided with a temperature indicator system (not shown) for measuring and displaying the temperature of the electrode 11 for delivery of radiofrequency current, and eventually, the temperature inside the balloon 6, through a detection signal from the temperature sensor 12 transmitted through the electric wire 43. Further, the radiofrequency generator 41 sequentially retrieves information on temperatures measured by the temperature indicator system to determine a level of a radiofrequency energy to be supplied through the electric wire 42 to between the electrode 11 for delivery of radiofrequency current and the counter electrode 13. Note that the electric wires 42, 43 are fixed along the inner tube 3 over the entire axial length of the inner tube 3.

According to the present embodiment, whilst the electrode 11 for delivery of radiofrequency current is used as a heating means for heating the inside of the balloon 6, it is not to be limited to any specific ones as long as it is capable of heating the inside of the balloon 6. For example, as substitute for the electrode 11 for delivery of radiofrequency current and the radiofrequency generator 41, there may be employed any one of couples of: an ultrasonic heating element and an ultrasonic generator; a laser heating element and a laser generator; a diode heating element and a diode power supply; and a nichrome wire heating element and a nichrome wire power supply unit.

Further, the catheter shaft 1 and the balloon 6 are made of such a heat resistant resin that can withstand heating without causing thermal deformation and the like when heating the inside of the balloon 6. The balloon 6 may take not only a spherical shape whose long and short axes are equal, but also any other shapes of any rotational bodies such as an oblate spherical shape whose short axis is defined as a rotation axis, a prolate spheroid whose long axis is defined as a rotation axis, or a bale shape. In any of these shapes, the balloon is made up of such an elastic member having compliance that deforms when it comes in close contact with an inside wall of a luminal organ.

When the balloon is subject to a positive pressure as described above, the amount of the coolant C to be discharged through gaps of the check valve 8 via the small holes 9 to the outside of the balloon 6, that is, discharge rate of the solution from the inside of the balloon 6 can be adjusted by the extent of in-and-out operation of the guide wire 10. FIGS. 1A and 1B illustrate such operation.

If the guide wire 10 within the inner tube 3 is allowed to slide toward a posterior direction, or toward the basal end in the axial direction thereof so as to arrange a distal end of the guide wire 10 in a position posterior to the discharge hole 3A to thereby open the discharge hole 3A, as illustrated in FIG. 1A, for example, then the discharge rate of the coolant C passing through the discharge hole 3A increases. In contrast, as illustrated in FIG. 1C, if the guide wire 10 is allowed to slide toward an anterior direction, or toward the distal end in the axial direction thereof so as to arrange the distal end of the guide wire 10 in a position anterior to the discharge hole 3A of the inner tube 3, then the discharge hole 3A is partially blocked, thereby decreasing the discharge rate of the coolant C passing through the discharge hole 3A. Consequently, as long as the valve 8 is open, discharge rate of the solution inside the balloon 6 can be easily adjusted through sliding operation of the guide wire 10.

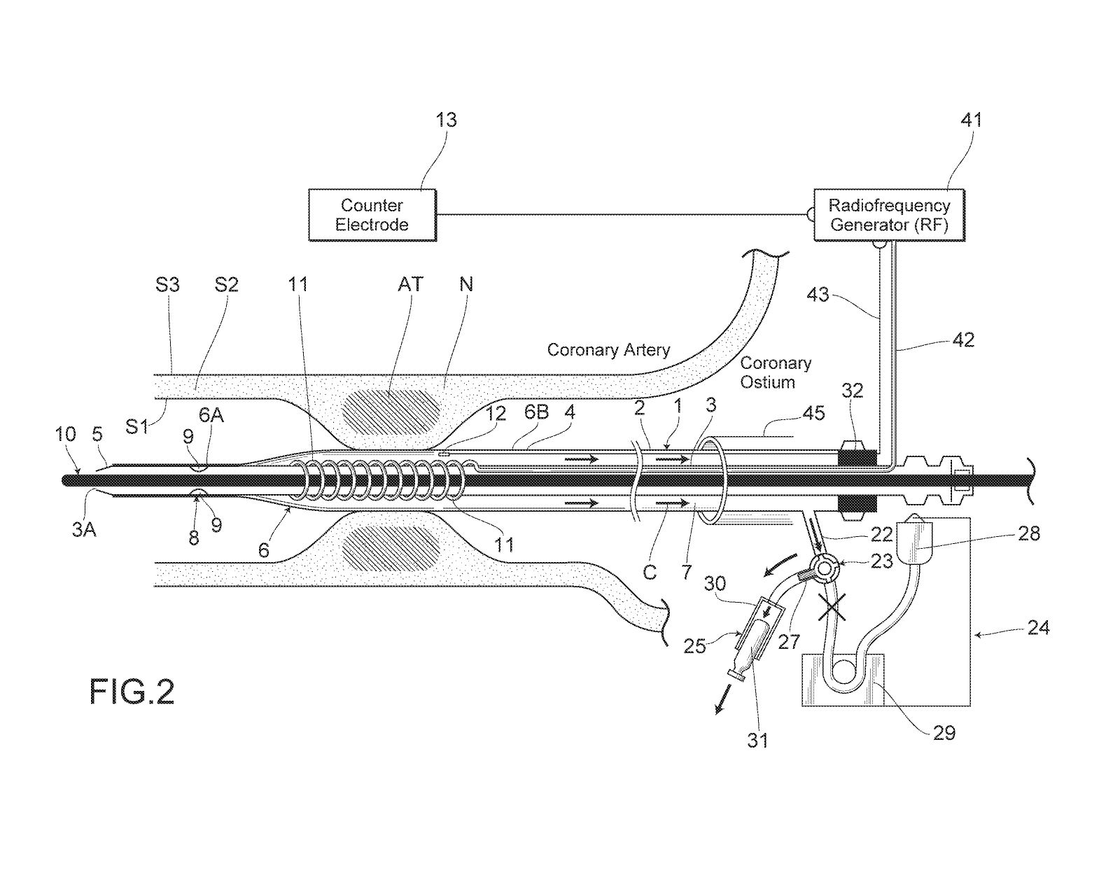

As to an implementing method of the above-discussed configuration, next is a description of the dilation procedures of coronary artery stenosis using the radiofrequency balloon catheter system according to the present embodiment with reference to FIGS. 2 to 4. In each of these figures, symbols S1, S2, and S3 respectively denote the intima, media and adventitia of a coronary artery. Symbol N denotes an artery stenosis site and symbol AT denotes atheroma. Here, FIGS. 1A to 1C should also be referred to because some anatomies are not illustrated in FIGS. 2 to 4.

Into the vicinity of a coronary ostium is intra-arterially inserted a guide sheath 45 through which the balloon catheter, including the catheter shaft 1 and the balloon 6, is further inserted into the coronary artery using the guide wire 10. At the posterior end of the catheter shaft 1, the syringe 25 is connected to the three-way cock 23 connected to the outlet of the solution transport path 7 that is communicated with the inside of the balloon 6 so as to bring the syringe 25 and the solution transport path 7 in communication with each other. Under that condition, if the plunger 31 is pulled back to strongly suction the inside of the balloon 6, the check valve 8 made up of the anterior neck 6A and the inner tube 3, is closed, thereby turning the inside of the balloon into a negative pressure, thus causing the balloon to be strongly contracted. As a result, the balloon 6 is allowed to be inserted into the artery stenosis site N, as illustrated in FIG. 2.

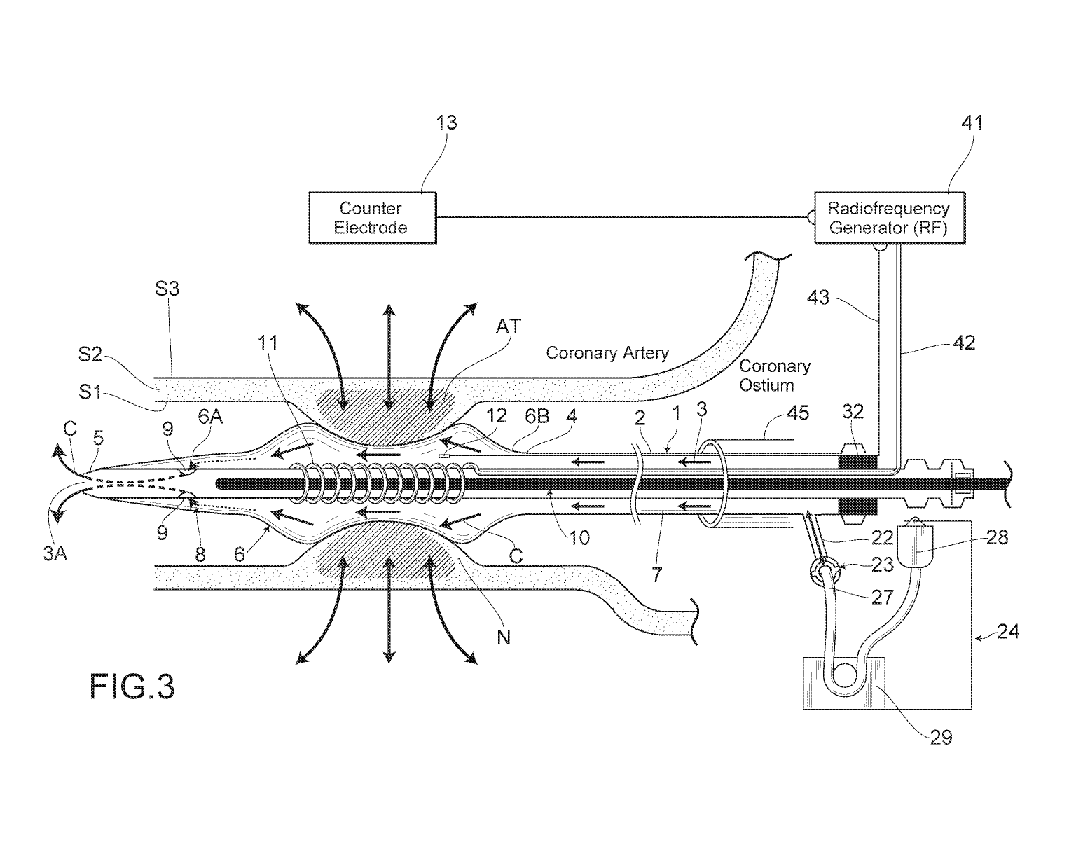

Next, as illustrated in FIG. 3, with the liquid transfusing pump 29 being connected to the communication tube 22 in communication with the solution transport path 7 such that the liquid transfusing pump 29 and the solution transport path 7 is brought into communication with each other through the three-way cock 23, there is initiated a delivery of radiofrequency current between the counter electrode 13 placed on the surface of a body and the electrode 11 for delivery of radiofrequency current provided within the balloon 6, using the radiofrequency generator 41, while the coolant C is being slowly injected into the balloon. Here, when injection rate of the coolant C is raised, the internal pressure of the balloon gets elevated, causing the balloon 6 to be inflated so that the check valve 8 is opened, thus allowing the coolant C to be discharged to the outside of the balloon 6 through the gaps of the check valve 8 via the small holes 9. If the artery stenosis site N, being in contact with the outer surface of the balloon 6, is not sufficiently dilated, then, the discharge hole 3A, serving as a hole of discharge outlet, is blocked using the guide wire 10 to thereby elevate the internal pressure of the balloon 6, or otherwise, radiofrequency output of the radiofrequency generator 41 is powered up in order to enhance the intensity of the electric field between the counter electrode 13 and the electrode 11 for delivery of radiofrequency current.

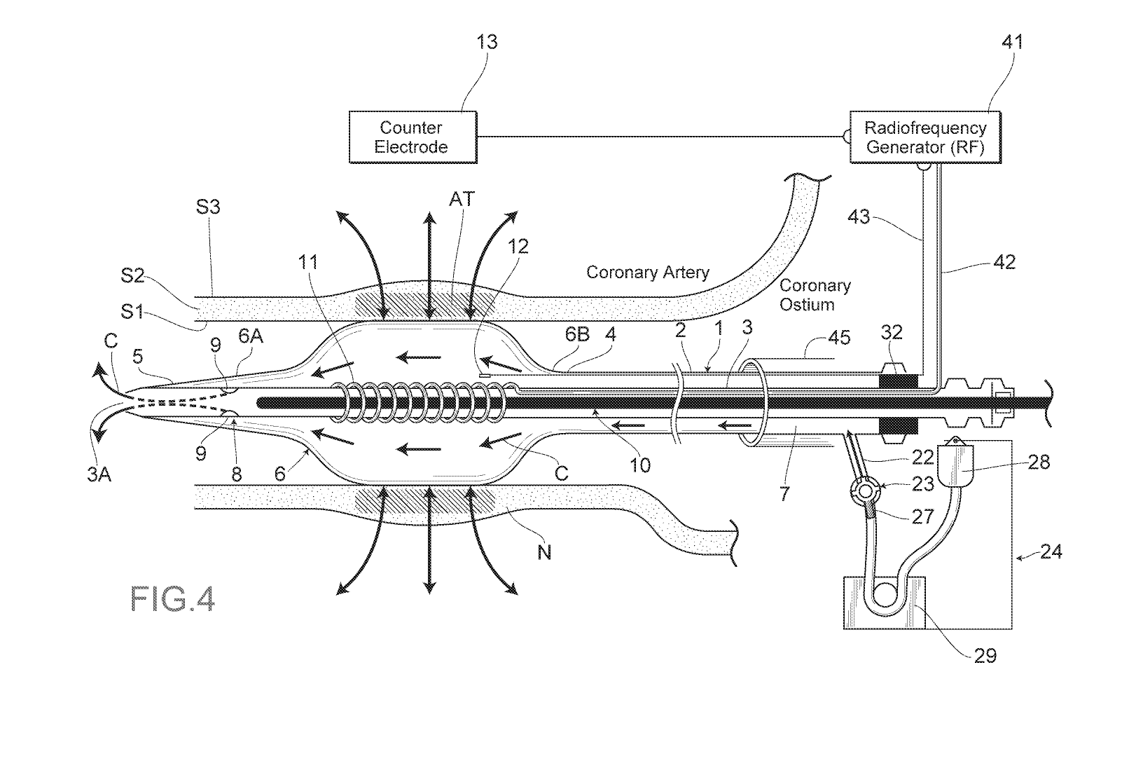

In this way, as illustrated in FIG. 4, if the artery stenosis site N gets sufficiently dilated, the radiofrequency generator 41 stops delivering the radiofrequency current, and then the coolant C serving as an in-balloon fluid is suctioned from the solution transport path 7 using the syringe 25 again to deflate the balloon 6, which is then removed out of the artery stenosis site N. After that, there will be performed a contrast study by way of the tip end of the catheter.

The radiofrequency balloon catheter system according to the present embodiment may be used not only for treatment of artery stenosis as explained above but also for treatment of, e.g., renal-artery stenosis and cerebral artery stenosis, or any other vascular stenoses which may occur all over the body. This system may also be applicable to treatment of stenoses at urethra, ureter, bile passage, or pancreas duct.

In summary, radiofrequency balloon catheters do not cause acute obstruction associated with vascular dissociation or recoil because angioplasty is performed while heating and dilating the stenosis site. Nevertheless, there still has a complication risk of restenosis associated with intimal proliferation. In order to prevent damages to an intima of a blood vessel, there have been proposed various balloon cooling methods in the past, but operability and performance thereof are not necessarily sufficient.

Then, according to the present invention, as described in regard to the embodiment of the present invention, the anterior neck 6A of the balloon 6 constituting the radiofrequency balloon catheter has a distal portion fixed to the inner tube 3, thus enabling it to easily pass through the stenosis site N. Further, the anterior neck 6A is provided in a manner covering the inner tube 3 so as to let both of them come close to each other to have a function as a check valve. Accordingly, without the need to change the profile thereof, inflation/deflation of the balloon 6 as well as discharge of the liquid inside the balloon 6 is allowed to be easily performed, thereby achieving enhanced performance and operability. That is, when the inside of the balloon 6 is suctioned by the syringe 25, the check valve 8 gets closed, causing the balloon 6 to be turned into a negative pressure. As the result, the balloon 6 gets deflated, enabling the same to easily pass through the stenosis site N. When the coolant C is injected, by the liquid transfusing unit 24, into the balloon 6 in order to inflate the same, the check valve 8 is opened to allow the in-balloon solution to be discharged through the small holes 9 bored through the inner tube 3 via the discharge hole 3A to the outside, thereby allowing the balloon 6 to be forcibly cooled. When a radiofrequency electric field is radiated from the electrode 11 for delivery of radiofrequency current arranged within the balloon 6, an arteriosclerosis site is heated and melted but the intima thereof remains protected by the cooling of the balloon 6. By enhancing the internal pressure within the balloon 6, stenosis sites get easily dilated without causing any dissection of the vessel.

As is apparent from the above, the radiofrequency balloon catheter system as proposed in the present embodiment has the catheter shaft 1 made up of the inner tube 3 and the outer tube 2. Between the distal end 4 of the inner tube 3 and the distal end 5 of the outer tube 2 is provided the resilient balloon 6 that is inflatable and deflatable. The anterior neck 6A of the balloon 6 has the distal portion that is fixed to the inner tube 3 while the proximal portion (or base section) of the anterior neck 6A covers the inner tube 3 to thereby define the check valve 8 such that the gap therebetween is open if the balloon 6 is at a positive pressure, while it is closed as they are arranged in contact with each other if the balloon 6 is at a negative pressure. Also, the transmural small holes 9 are bored through the inner 3 tube serving as a valve seat for the check valve 8. Within the balloon 6 is arranged the electrode 11 for delivery of radiofrequency current, which is connected to the radiofrequency generator 41 via the electric wire 42. The solution transport path 7 that is defined by the outer tube 2 and the inner tube 3 and is in constant communication with the inside of the balloon 6, is connected to the liquid transfusing pump 29 serving as a liquid feed pump for feeding the coolant C. Further, into the hollow inner tube 3 is insertable the guide wire 10 that comes in and out from the discharge hole 3A at the distal end of the inner tube 3.

The above-described schematic configurations are illustrated in FIG. 1A. Further, as illustrated in FIG. 1C, when the coolant C inside the balloon 6 is suctioned through the catheter shaft 1, the check valve 8 defined by the anterior neck 6A of the balloon 6 and the inner tube 3 is closed, causing the inside of the balloon 6 to be turned into a negative pressure. Also, as illustrated in FIG. 2, when the coolant C inside the balloon 6 is suctioned, the balloon 6 is deflated and thus inserted into the artery stenosis site N.

As illustrated in FIG. 3, when the coolant C is injected into the balloon 6 through the catheter shaft 10, the balloon 6 becomes inflated to cause the check valve 8 defined by the inner tube 3 and the proximal portion of the anterior neck 6A to be opened, letting the coolant C pass through the small holes 9 bored through the inner tube 3, so that the coolant C is discharged through the distal end of the inner tube 3 to the outside of the balloon 6, thereby cooling the balloon 6 as the balloon 6 itself serves as a path for the coolant C. Discharge rate of the coolant C depends on injection rate of the coolant to be injected into the balloon 6, and further on the elasticity and/or shape of the anterior neck 6A of the balloon 6 serving as a valving element. Further, by allowing the guide wire 10 within the inner tube 3 to slide to change the extent of “overlap” between the inner tube 3 and the discharge holes 9, there can be adjusted the discharge rate of the coolant C coming out of the balloon 6. If the guide wire 10 is inserted up to a position beyond the small holes 9 of the inner tube 3, then, a route of discharge within the inner tube 3 is subjected to an increased resistance, thus decreasing discharge rate of the coolant C. Namely. discharge rate of the coolant C, coming out of a distal end of the balloon catheter, can be adjusted through manipulation of the guide wire 10, that is, when the guide wire 10 is inserted up to the distal end of the inner tube 3, then the route of discharge is subjected to an increased resistance, resulting in a decreased discharge rate of the coolant C. In contrast to this, when the guide wire 10 is pulled back behind the distal end of the inner tube 3, then the route of discharge is subjected to a decreased resistance, resulting in an increased discharge rate of the coolant C, as illustrated in FIGS. 1A and 1B.

Concurrently therewith, upon delivery of radiofrequency current, a radio frequency electric field is radiated uniformly from the electrode 11 for delivery of radiofrequency current arranged inside the balloon 6, thereby allowing the balloon 6 to dilate the stenosis site N while heating the same. Also, when a coolant is injected into the balloon 6 simultaneously therewith, the check valve 8 is opened, allowing the coolant C to be discharged from the lumen of the inner tube 3 to the outside through the small holes 9 of the inner tube serving as a valve seat, thereby cooling the balloon 6.

Such cooling system for the balloon 6 enables the intima 51 to be protected against heating, as illustrated in FIG. 4.

According to the present embodiment, there can be provided a radio frequency balloon catheter system enabling a balloon catheter thereof to easily pass through the stenosis site N to dilate the stenosis while applying radiofrequency heat to the same, with the intima 51 being protected by a cooling effect achieved by an appropriate perfusion inside the balloon 6.

Next, there will be described other various preferred modifications to the above-described radio frequency balloon catheter system.

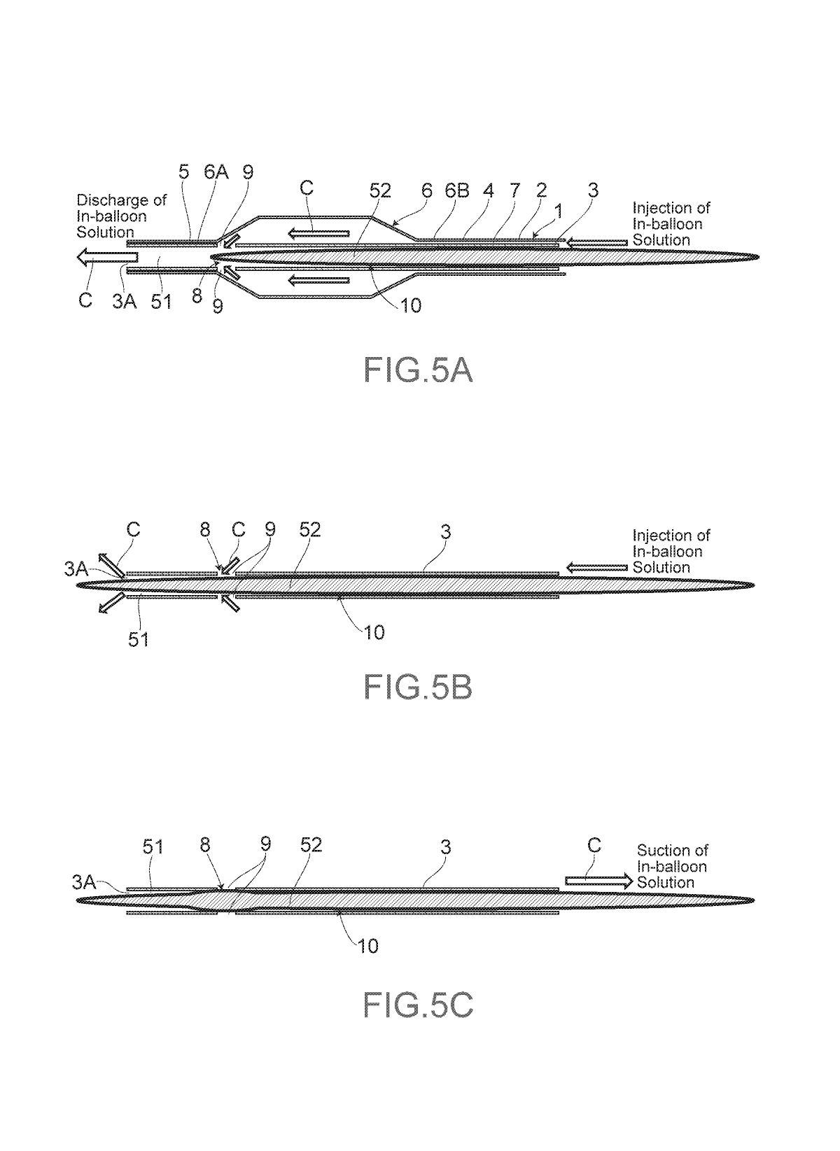

FIGS. 5A to 5C illustrate a first modified embodiment where the guide wire 10 that is rich in resiliency is employed as a valving element of the check valve 8. It is to be noted herein that FIG. 5A should also be referred to when referring to FIGS. 5B and 5C because the outer tube 2 and the balloon 6 are not illustrated in these figures. As illustrated here in FIG. 5A, the anterior neck 6A of the balloon 6 is entirely fixed to the outer surface of the inner tube 3. At the distal end portion 5 of the inner tube 3 is provided a hollow nozzle 51 whose distal end tip is opened to form the discharge hole 3A. Further, at the distal side portion of the inner tube 3 serving as a valve seat for the check valve 8 within the balloon 6, there are provided the small holes 9 bored through a sidewall of the inner tube 3 in order to discharge the fluid therefrom.

A coating layer 52 made of a resilient material is formed on the surface of the guide wire 10 that is insertable through the inner tube 3, and is configured to be expanded or contracted by an external force. Further, the guide wire 10 has a distal end portion having such a tapered shape that is gradually tapered toward a distal end. Owing to this configuration, when the distal end of the guide wire 10 slides forward, the guide wire 10 comes into contact with and conforms with the lumen of the inner tube 3 having the small holes 9 formed therein. The other configurations are identical with those in the above embodiment.

According to this modified embodiment, a part of the inner tube 3 where the small holes 9 are formed is contacted by the resilient portion of the guide wire 10 to thereby define the check valve 8. Accordingly, as illustrated in FIG. 5A, when the distal end of the guide wire 10 is arranged behind the discharge hole 3A of the inner tube 3 so that the discharge hole 3A is in an opened state, there are provided a large gap on the periphery of the small holes 9 of the inner tube 3. Owing to this configuration, the in-balloon solution injected through the solution transport path 7 will hardly be blocked by the guide wire 10, and be guided through the small holes 9 into the interior of the nozzle 51 from which the solution is allowed to be discharged through the discharge hole 3A to the outside of the balloon 6.

In this modified embodiment as well, when the inside of the balloon 6 is under a positive pressure while the coolant C is being pumped out through the solution transport path 7 into the balloon 6, discharge rate of the coolant C to be discharged to the outside of the balloon 6 can be freely adjusted by the extent of in-and-out operation of the guide wire 10. As illustrated in FIG. 5B, when the guide wire 10 is allowed to slide forward with its distal end being arranged anterior to the discharge hole 3A, the coating layer 52 of the guide wire 10, serving as a resilient portion thereof, becomes deformed and contracted to thereby open the check valve 8, thus allowing the coolant C to be discharged from gaps between the inner tube 3 and the guide wire 10 through the discharge hole 3A to the outside of the balloon 6. At this moment, since the discharge hole 3A is partially blocked by the guide wire 10, the discharge rate of the coolant C passing through the discharge hole 3A will be lower than that as illustrated in FIG. 5A. Further, since the distal end portion of the guide wire 10 is tapered, the more the guide wire is allowed to slide forward, the wider the area to be blocked by the discharge hole 3A becomes, leading to a reduced gap between the inner tube 3 and the guide wire 10, eventually leading to a gradually decreased discharge rate of the coolant C. In this way, when the check valve 8 is in an opened state, discharge rate of the solution inside the balloon 6 can be easily adjusted through the sliding operation of the guide wire 10.

On the other hand, as illustrated in FIG. 5C, when the coolant C, serving as an in-balloon solution of the balloon 6, is suctioned through the solution transport path 7, its suction power causes the inside of the balloon to come under negative pressure, thereby causing the coating layer 52 of the guide wire 10, serving as a resilient portion, to be deformed and expanded to close the check valve 8, thus closing the nozzle 51 of the inner tube 3 to have the balloon 6 forcibly deflated. Note that these technical features can be applied to other embodiments or modifications.

According to the present modified embodiment, there is provided the radiofrequency balloon catheter system, in which the catheter shaft 1 is made up of the inner tube 3 and the outer tube 2; between the distal end 5 of the inner tube 3 and the distal end 4 of the outer tube 2 is provided the resilient balloon 6 that is inflatable and deflatable; the anterior neck 6A of the balloon 6 is fixed to the inner tube 3 arranged within the balloon 6; the one or more small holes 9 bored through the distant portion of the inner tube 3 within the balloon 6; inside the inner tube 3 is interposed the guide wire 10 whose surface is coated with the coating layer 52 of resilient material; the small holes 9 and the guide wire 10 are contacted by each other to define the check valve 8; within the balloon 6 is arranged the electrode 11 for delivery of radiofrequency current, which is then connected to the radiofrequency generator 41 via the electric wire 42; the solution transport path 7 is defined by the outer tube 2 and the inner tube 3, and connected to the liquid transfusing pump 29 serving as a liquid feed pump for feeding the coolant C, and the solution transport path 7 is constantly in communication with the inside of the balloon 6.

In this way, according to this modified embodiment, there can be ensured a discharge route for the coolant, passing from the balloon 6 to the inside of the inner tube 3, by boring the small holes 9 through the inner tube 3 within the balloon 6 so that the inner tube 3 may serve as a valve seat, while the guide wire 10 having such a resiliency owing to the coating layer 52 that expands and contracts in response to a pressure is allowed to serve as a valving element, whereby the check valve 8 is closed to deflate the balloon when the inside of the balloon 6 is under negative pressure, while the check valve 8 is opened to discharge the coolant when the inside of the balloon is under positive pressure, as illustrated in FIGS. 5A to 5C. Like in the above described embodiments, there can be provided a radio frequency balloon catheter system enabling a stenosis to be dilated while heating the same, with an intima Si being protected through the perfusion inside the balloon while delivering a radiofrequency current thereto.

Also, the guide wire 10 has such a tapered distal end that conforms to a lumen of the inner tube 3. The guide wire 10, having a role to adjust the discharge rate of a coolant by closing the lumen of the inner tube 3, has such a tapered distal end that is conformable to the lumen of the inner tube 3, thereby enhancing the function thereof.

FIG. 6 illustrates a second modified embodiment where electrodes 15a, 15b and a pressure sensor 16 are incorporated into the system in addition to the temperature sensor 12. As shown in this figure, the temperature sensor 12 is provided within a distal portion provided as a tip end section of the inner tube 3 so as to enable temperature measurement of the coolant C that are to be discharged through the discharge hole 3A of the inner tube 3. Further, around the inner tube 3 within the balloon 6, there is put a pressure sensor 16 which enables internal pressure measurement inside of the balloon 6. Furthermore, outside the balloon 6, there are provided the aforesaid electrodes 15a and 15b that are respectively arranged on the distal end portion 5 of the inner tube 3 and in the vicinity of the distal end portion 4 of the outer tube 2.

Outside the balloon shaft 1 are arranged an electric impedance measuring potential amplifier 61, a radiofrequency filter 62 and a pressure gauge 63. The electric impedance measuring potential amplifier 61 is connected to the electrodes 15a, 15b, arranged at the front and rear of the balloon 6, respectively through the electric wires 65 and 66, allowing a weak current to flow between the electrodes 15a, 15b, thereby measuring an electric impedance obtained from the voltage value at that time as an electric impedance thereof surrounding the balloon 6, thereby providing the same with a function serving as an electric impedance measuring equipment. Further, the electric impedance measuring potential amplifier 61 has a function to serve as an amplifier for amplifying a far-field potential obtained from the electrodes 15a, 15b and recording that potential, thereby tracking the abrasion progress of the target tissue through monitoring the changes in the electric impedance and potential waveform. Also, the radiofrequency filter 62 is incorporated into the electric circuit for measurement that is composed of the electrodes 15a, 15b, the electric impedance measuring potential amplifier 61 and the electric wires 65, 66 in order to eliminate the influence of the radiofrequency noise generated from the radiofrequency generator 41. In the same way as the foregoing electric wires 42, 43, the electric wires 65, 66 are fixed along the inner tube 3 over the entire axial length of the inner tube 3.

Further, inside the balloon 6 is provided a pressure sensor 16 that outputs detection signals in response to the pressure received on its input surface, and is electrically connected to a pressure gauge 63 through an electric wire 68 provided within the catheter shaft 1. The electric wire 68 is fixed along the inner tube 3 over the whole length thereof extending in an axial direction thereof. As illustrated in FIG. 6, the electric wire 68 is provided outside the electrode 11 for delivery of radiofrequency current. Alternatively, the electric wire 68 may be interposed in the electrode 11 for delivery of radiofrequency current that is provided in a coiled fashion.

The pressure gauge 63 is configured to measure, through detection signals sent out from the pressure sensor 16 via the electric wire 68, a pressure applied from the balloon 6 to a target site, that is, a pressing force, as a degree of pressure applied from the balloon 6 against the target tissue, and then to display the pressure thus measured. The pressure gauge 63 is arranged outside the balloon catheter 21 along with the radiofrequency generator 41. Preferably, the electric impedance measuring potential amplifier 61 and the radiofrequency generator 41 may be electrically connected with each other so as to allow the measurement outcomes of electric impedance or potential waveform, measured by the electric impedance measuring potential amplifier 61, to be taken into the radiofrequency generator 41. Moreover, the pressure gauge 63 and the radiofrequency generator 41 may be configured to be electrically connected with each other so as to allow the measurement outcomes of pressure, measured by the pressure gauge 63, to be taken into the radiofrequency generator 41. In that case, the radiofrequency generator 41 is allowed to serve as a device for monitoring an ablation progress, enabling a centralized administrative monitoring of not only a temperature of the balloon 6 and a period of an energization to the electrode 11 for delivery of radiofrequency current, but also an electric impedance around the balloon 6, waveforms of the electric potentials, and a pressing force from the balloon 6 against the tissue. The present embodiment shares common features with the foregoing embodiments except the features described above.

Then, when the balloon 6 is in a state of being inflated, the surrounding space of the pressure sensor 16 is filled with the coolant C while the stream of the coolant C is constantly flowing through the gap toward the outside of the balloon 6. Nevertheless, the directional pressure sensor 16 is hardly affected by the pressure associated with such stream of the coolant C. The pressing forces developed when pressing the balloon 6 against the target site, e.g., vascular stenosis site N, are to be transmitted from the front surface of the membrane of the balloon 6 to the input surface of the pressure sensor 16 via the coolant C provided thereinside. For this reason, the pressure sensor 16 becomes highly directive, thereby allowing one to accurately monitor the pressing force from the balloon 6 against the tissue without being influenced by the stream of coolant C inside the balloon 6.

Further, detection signals from the temperature sensor 12 are sent through the electric wire 43 to the radiofrequency generator 41 provided with a thermometer or temperature meter. In response to this, the radiofrequency generator 41 measures the temperature of a perfusate discharged from the discharge hole 3A of the inner tube 3, i.e., from the balloon catheter. Based on the result of these measurements, the temperature of the perfusate can be maintained at a preset temperature through the regulation of the electric current to be applied to the electrode 11 for delivery of radiofrequency current. As described above, if the temperature sensor 12 is provided within the balloon 6, there can be received a detection signal of the temperature sensor by the radiofrequency generator 41 to thereby monitor the temperature inside the balloon 6 along with the monitoring results of the pressure sensor 16, thereby enabling one to make sure the effectiveness of the ablation against the target tissue.

Further, the electric impedance measuring potential amplifier 61 allows a weak electric current to flow across the electrodes 15a and 15b via the electric wires 65 and 66 to thereby monitor the electric impedance and far-field potential around the balloon 6, thereby enabling tracking of the ablation progress against the target tissue.

That is, according to this modification, to the distal end of the inner tube 3 is attached the temperature sensor 12 for measuring temperature of a perfusate coming out of the balloon 6. In this case, by virtue of the temperature sensor 12 attached to the distal end of the inner tube 3, it becomes possible to measure a temperature of a perfusate discharged from the balloon catheter by the radiofrequency generator 41. If the temperature is kept at 45 degrees C. or below, it is possible to reduce peripheral vascular disorder to minimum, while if the temperature is kept at more than 45 degrees C., it is possible to perform hyperthermic treatment to a peripheral perfusion area.

Alternatively, there may be arranged the temperature sensor 12 and the pressure sensor 16 within the balloon 6, such that the temperature sensor 12 may be connected via the electric wire 43 to the radiofrequency generator 41 including the temperature measurement device, while the pressure sensor 16 may be connected to the pressure gauge 63, serving as a pressure measurement device, through a different electric wire 68. By virtue of the temperature sensor 12 and the pressure sensor 16 respectively provided within the balloon 6, there can be monitored a temperature within the balloon 6 and a pressing force of the balloon 6 against tissues, thus enabling one to make sure the effectiveness of ablation against the target tissue.

Further, according to this modified embodiment, there are provided the electrodes 15a, 15b on the anterior and posterior portions of the balloon 6 on the catheter shaft 1, in which the electrodes 15a, 15b are connected via the electric wires 65, 66 to the electric impedance measuring potential amplifier 61 serving as an impedance measurement device. Owing to these electrodes 15a, 15b being arranged in the anterior and posterior portions of the balloon 6, there can be monitored an impedance around the balloon 6, thereby enabling tracking of the ablation progress against the target tissue.

It should be noted that the number of the small holes 9 is preferably set to be 1 to 10 throughout the embodiments and modified embodiments described above. The rate of perfusion flowing through the balloon 6 can be finely adjusted by increasing the number of the small holes 9 bored through the inner tube 3 serving as a valve seat.

The membrane constituting the balloon 6 is preferably made of a conductive film or a porous film. Hence, electric conductivity of the balloon membrane can be enhanced to facilitate irradiation of radiofrequency electric field onto the surrounding tissue.

The present invention shall not be limited to the embodiments described above, and various modified embodiments are possible within the scope of the present invention. The radiofrequency balloon catheter system of the present invention can be used for dilation of stenosis sites in hollow organs such as urethra, ureter, pancreas duct, trachea, esophagus, and intestine in addition to blood vessel and bile passage. Further, the catheter shaft 1, the balloon 6 and the guide wire 10 may have other various shapes conforming to the sites to be treated, and shall not be limited to those described in the foregoing embodiments.

1. A radiofrequency balloon catheter system comprising:
a catheter shaft comprising an inner tube and an outer tube;
a resilient balloon that is inflatable and deflatable and provided between distal ends of the inner tube and the outer tube, said balloon including an anterior neck covering said inner tube, said anterior neck having a distal portion fixed to said inner tube and a proximal portion contacted by said inner tube to thereby define a check valve;
one or more transmural small holes bored through a part of said inner tube that serves as a valve seat for said check valve;
an electrode for delivery of radiofrequency current provided within the balloon;
a radiofrequency generator connected to the electrode for delivery of radiofrequency current via a connecting wire within said catheter shaft;
a solution transport path defined by the outer tube and the inner tube, said solution transport path being in communication with an inside of the balloon, and connected to a liquid feed pump for feeding a coolant; and
a guide wire insertable into said inner tube.

2. The radiofrequency balloon catheter system according to claim 1, wherein the number of said one or more transmural small holes is 1 to 10.

3. The radiofrequency balloon catheter system according to claim 1, wherein said guide wire has such a tapered distal end that conforms to a lumen of said inner tube.

4. The radiofrequency balloon catheter system according to claim 1, wherein a temperature sensor for measurement of a perfusate temperature is attached to the distal end of said inner tube.

5. The radiofrequency balloon catheter system according to claim 1, wherein a temperature sensor and a pressure sensor are installed within said balloon and are respectively connected to a temperature measurement device and a pressure measurement device via a connecting wire.

6. The radiofrequency balloon catheter system according to claim 1, wherein an electrode is installed in front and back of said balloon on said catheter shaft, and the electrode is connected to an impedance measurement device via a connecting wire.

7. The radiofrequency balloon catheter system according to claim 1, wherein said balloon is made up of a film that is either a conductive film or a porous film,

8. A radiofrequency balloon catheter system comprising:
a catheter shaft comprising an inner tube and an outer tube;
a resilient balloon that is inflatable and deflatable and provided between distal ends of the inner tube and the outer tube, said balloon including an anterior neck fixed to said inner tube;
one or more small holes bored through a distant portion of said inner tube within said balloon;
a guide wire provided within said inner tube, said guide wire having a surface coated with a resilient material;
a check valve defined by said one or more small holes and said guide wire being contacted by each other;
an electrode for delivery of radiofrequency current provided within the balloon;
a radiofrequency generator connected to the electrode for delivery of radiofrequency current via a connecting wire within said catheter shaft; and
a solution transport path defined by the outer tube and the inner tube, said solution transport path being in communication with an inside of the balloon, and connected to a liquid feed pump for feeding a coolant.

9. The radiofrequency balloon catheter system according to claim 8, wherein the number of said one or more small holes is 1 to 10.

10. The radiofrequency balloon catheter system according to claim 8, wherein said guide wire has such a tapered distal end that conforms to a lumen of said inner tube.

11. The radiofrequency balloon catheter system according to claim 8, wherein a temperature sensor for measurement of a perfusate temperature is attached to the distal end of said inner tube.

12. The radiofrequency balloon catheter system according to claim 8, wherein a temperature sensor and a pressure sensor are installed within said balloon and are respectively connected to a temperature measurement device and a pressure measurement device via a connecting wire.

13. The radiofrequency balloon catheter system according to claim 8, wherein an electrode is installed in front and back of said balloon on said catheter shaft, and the electrode is connected to an impedance measurement device via a connecting wire.

14. The radiofrequency balloon catheter system according to claim 8, wherein said balloon is made up of a film that is either a conductive film or a porous film.
